(19) United States
(12) Patent Application Publication
(10) Pub. No.: US 2026/0069808 A1
(43) Pub. Date: Mar. 12, 2026

(54) TUBE INTERFACE GUARD FOR EXTUBATION AND RANGING

(71) Applicant: Board of Supervisors of Louisiana State University and Agricultural and Mechanical College, Baton Rouge, LA (US)

(72) Inventors: Jonathan S. Alexander, Shreveport, LA (US); Giovanni Solitro, Shreveport, LA (US); Donald Sorrells, Shreveport, LA (US)

(21) Appl. No.: 19/326,419

(22) Filed: Sep. 11, 2025

Related U.S. Application Data
(60) Provisional application No. 63/693,295, filed on Sep. 11, 2024.

(51) Int. Cl. A61M 16/04

(52) U.S. Cl. CPC A61M 16/0465; A61M 16/0488; A61M 2205/18; A61M 2205/332; A61M 2205/3592; A61M 2205/6054; A61M 2230/63

(57) ABSTRACT

A device comprising an arcuate face bar, wherein the arcuate face bar is configured for attachment to an endotracheal tube deployed upon a patient, at least two radio frequency identification (RFID) probes secured to opposing terminal ends of the arcuate face bar, wherein the attachment of the arcuate face bar to the endotracheal tube positions the at least two RFID probes at locations in proximity to respective first and second RFID tags affixed to skin of the patient, the RFID probes configured to communicate an occurrent of an event when one of the at least two RFID probes detects a received signal strength of a respective RFID tag below a threshold value.

### RELATED APPLICATION

This application claims the benefit of US Application No. 63/693,295, filed Sep. 11, 2024.

### TECHNICAL FIELD

Embodiments are described herein relating to endotracheal tube monitoring systems.

### BACKGROUND

In healthcare management, an alarming threat to the safety of ventilated patients is Unplanned Extubation or Tube Migration. This occurs when a patient or external force inadvertently removes or moves a poorly secured breathing tube from the airway (1, 2, 3).

Annually, Unplanned Extubation adversely affects over 121,000 patients, resulting in more than 36,000 cases of ventilator-associated pneumonia, over 33,000 preventable deaths, and an excess of $4.9 billion in avoidable healthcare costs. The median incidence rate of Unplanned Extubation stands at 7.3% for all ventilated ICU patients (1, 2, 3).

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

A device is described herein comprising an arcuate face bar, wherein the arcuate face bar is configured for attachment to an endotracheal tube deployed upon a patient, at least two radio frequency identification (RFID) probes secured to opposing terminal ends of the arcuate face bar, wherein the attachment of the arcuate face bar to the endotracheal tube positions the at least two RFID probes at locations in proximity to respective first and second RFID tags affixed to skin of the patient, the RFID probes configured to communicate an occurrent of an event when one of the at least two RFID probes detects a received signal strength of a respective RFID tag below a threshold value.

In embodiments, at least one of tube migration, movement of the patient, an external force, or detachment of the arcuate face bar from the endotracheal tube causes reorientation of the at least two RFID probes relative to the respective RFID tags.

In embodiments, the at least two RFID probes comprise a first RFID probe.

In embodiments, the attachment locates the first RFID probe in proximity to the first RFID tag affixed to cheek bone skin surface adjacent the patient's auris dexture ear.

In embodiments, the at least two RFID probes comprises a second RFID probe.

In embodiments, the attachment locates the second RFID probe in proximity to the second RFID tag affixed to cheek bone skin surface adjacent the patient's auris sinistra ear.

In embodiments, the at least two RFID probes comprises a third RFID probe.

In embodiments, the attachment locates the third RFID probe in proximity to a third RFID tag at least one of affixed to or integrated into the endotracheal tube without obstructing airflow therethrough.

In embodiments, the attachment locates the third RFID probe in proximity to a third RFID tag affixed to a skin surface of the patient's parioral region.

In embodiments, the location of the third RFID tag comprises skin surface adjacent to the patient's lower central lip.

In embodiments, the location of the third RFID tag comprises skin surface adjacent to the patient's upper central lip.

In embodiments, the at least two RFID probes emit a radio frequency signal into an environment of the respective RFID tag.

In embodiments, the respective RFID tag's antenna captures this electromagnetic energy, which is used to power up a microchip inside the tag.

In embodiments, the powered microchip modulates its stored identification data and sends it back to the respective at least two RFID probes as a return signal.

In embodiments, the return signal comprises backscatter communication.

In embodiments, the at least two respective RFID readers receive and decode the signal to identify the respective tag and process the information.

In embodiments, the processing comprises detecting the received signal strength of the signal.

In embodiments, the attachment comprises securing a central portion of the arcuate face bar to the endotracheal tube.

In embodiments, the securing comprises use of an adhesive.

In embodiments, the securing comprises use of a mounting clamp.

### BRIEF DESCRIPTION OF THE FIGURES

FIG. 1 shows a top view of a TIGER guard mechanical structure, under an embodiment.

FIG. 2 shows a mounting clip, under an embodiment.

FIG. 3 shows an RFID microcontroller communicatively coupled with three RFID probes, under an embodiment.

FIG. 4A shows an RFID microcontroller, under an embodiment.

FIG. 4B shows an RFID probe, under an embodiment.

FIG. 4C shows an RFID probe, under an embodiment.

FIG. 4D shows an RFID probe, under an embodiment.

FIG. 5 shows deployment of a monitoring guard, under an embodiment.

FIG. 6 shows deployment of a monitoring guard, under an embodiment.

FIG. 7 shows deployment of a monitoring guard, under an embodiment.

FIG. 8 shows deployment of a monitoring guard, under an embodiment.

FIG. 9 shows an integrated RFID microcontroller and guard structure, under an embodiment.

FIG. 10 shows an integrated RFID microcontroller and guard structure, under an embodiment.

FIG. 11 shows an integrated RFID microcontroller and guard structure, under an embodiment.

### DETAILED DESCRIPTION

The prevalence of Unplanned Extubation or Tube Migration with respect to ventilated patient is deemed unacceptable. TIGER, short for Tube Interface Guard for Extubation and Ranging, is a practical solution designed to address a critical concern in healthcare - the accidental movement or dislodgment of endotracheal tubes during mechanical ventilation. Using RFID technology, TIGER constantly monitors the position of the tube face attachment and triggers an alarm if it moves beyond a predefined safe distance. TIGER operates through a straightforward yet effective mechanism. The endotracheal tube is equipped with an RFID tag, and its proximity is continuously monitored by an RFID probe. This probe is securely integrated into the face bar of the ventilation system. In the event of any undesired movement or dislodgment of the tube, the RFID probe promptly detects the change in proximity and triggers a simple alarm circuit so that a nurse or other staff member can quickly correct the problem. One or more RFID probe/tag combinations may be used as further described below.

Although there are numerous RFID technologies, passive RFID tags stand out as the most common and cost-effective option. With no onboard power source and a long lifespan, passive RFID is an attractive option for use in the TIGER guard.

Passive RFID tags are the most common and widely used type of RFID tag. Unlike active or semi-active tags, passive tags don't contain a battery. Instead, they rely entirely on the radio waves emitted by an RFID reader to power up and operate. Rather, a passive RFID tag is a battery-free RFID device that gets activated when it comes into range of a reader. The electromagnetic field from the reader powers the tag's chip, allowing it to transmit stored data.

RF tags are categorized by the frequency upon which they operate:

Low Frequency (LF, 125 kHz)—Short read range, good penetration. Common in pet microchips and basic keycards.

High Frequency (HF, 13.56 MHz)—Widely used in library cards, transit passes, and NFC-based systems.

Ultra-High Frequency (UHF, 860-960 MHz)—Longer read range (up to 10 meters) and faster data transfer. Ideal for retail inventory, asset tracking, and logistics.

Understanding how passive RFID tags operate is essential for grasping their benefits and limitations. While they lack an internal power source, their design allows them to communicate efficiently using energy from the RFID reader.

The RFID reader or probe and RF tag interact as follows:
the RFID reader emits a radio frequency signal into the environment
the tag's antenna captures this electromagnetic energy, which is used to power up the microchip inside the tag
once powered, the chip modulates its stored data (like a unique ID) and sends it back to the reader
this return signal is reflected back to the reader using a technique called backscatter communication
the RFID reader receives and decodes the signal to identify the tag and process the information

As indicated above, passive RFID tags do not contain a battery. Instead, all operating power is harvested from the RFID reader's transmitted signal. This limits their read range but also keeps the tag cost low and lifespan long. Unlike active tags that actively transmit signals, passive tags use backscatter to reflect the reader's signal back, embedding their data in the reflection. This method is highly energy-efficient and ideal for basic identification tasks.

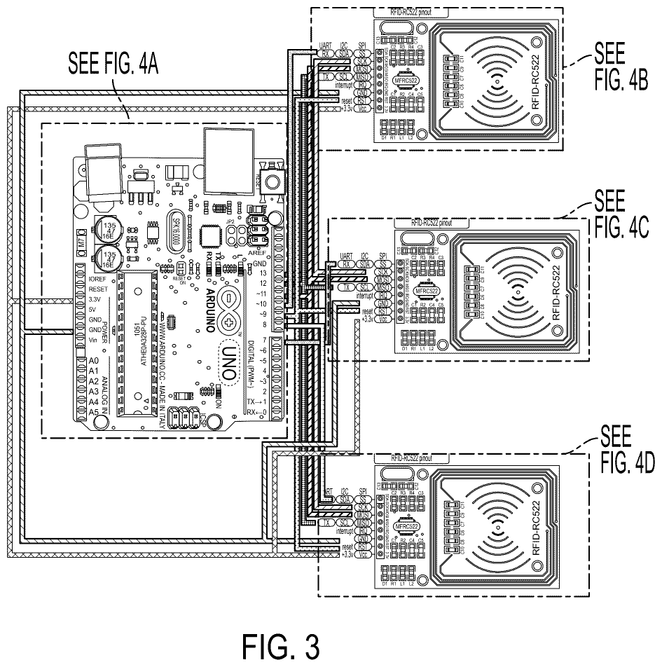

FIG. 3 shows an RFID microcontroller communicatively coupled with three RFID antenna/probes, under an embodiment. (The coupling as shown is wired but may also be wireless). As further described below, the antennas/probes are secured to a guard device which locate the antennas/probes in proximity to RF tags. Under this embodiment, Arduino antennas/probes (coupled to an Arduino microcontroller) are used to implement the TIGER guard monitoring system, but embodiments are not so limited and alternative RFID reader/tag systems may be used.

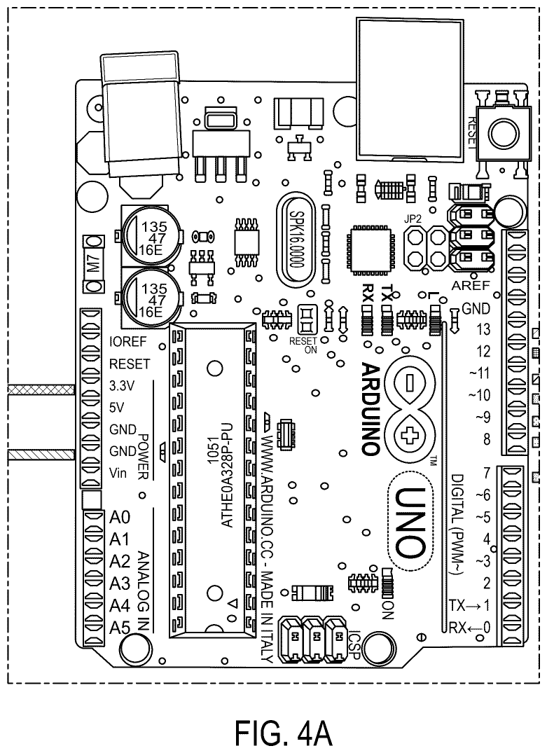

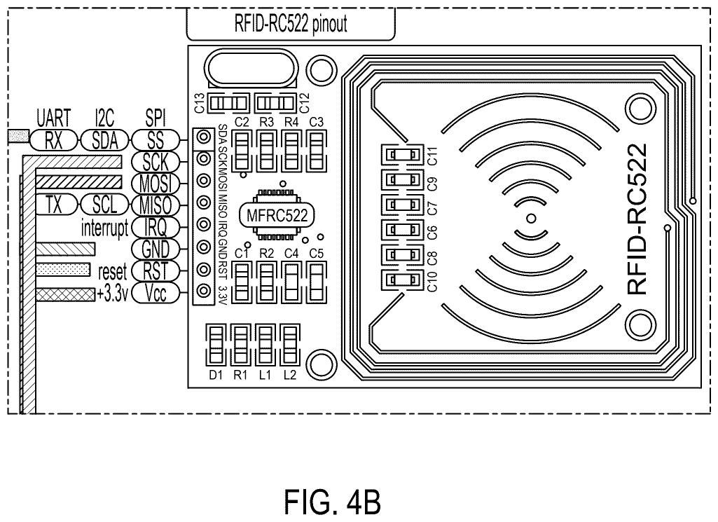

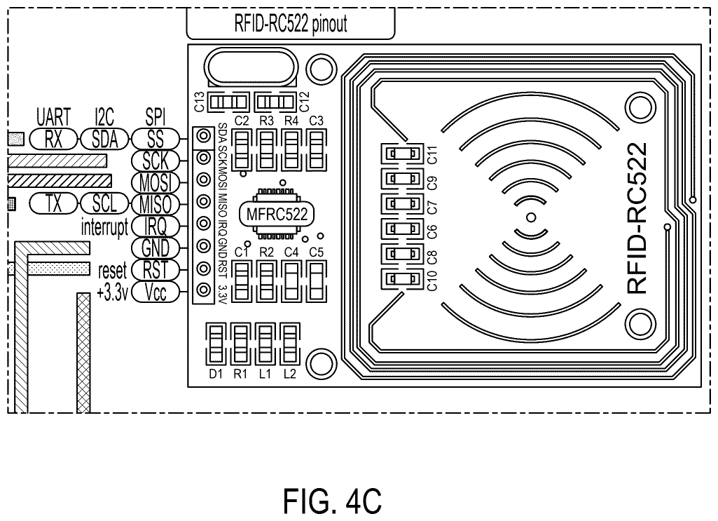

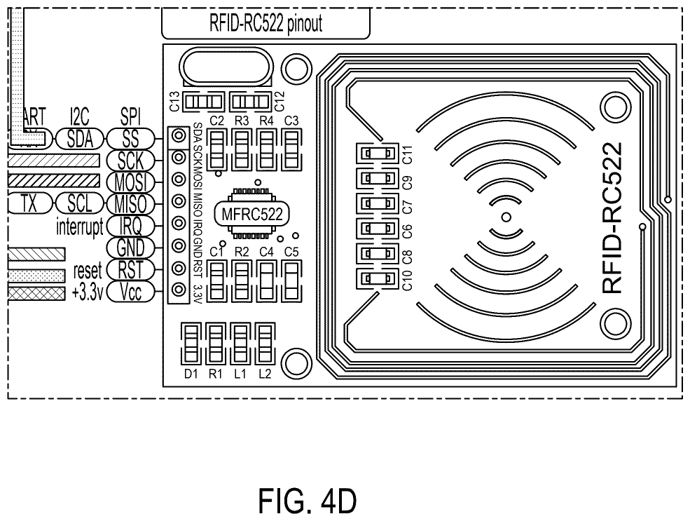

FIG. 4A shows the RFID microcontroller, under an embodiment.

FIG. 4B shows the RFID probe, under an embodiment.

FIG. 4C shows the RFID probe, under an embodiment.

FIG. 4D shows the RFID probe, under an embodiment.

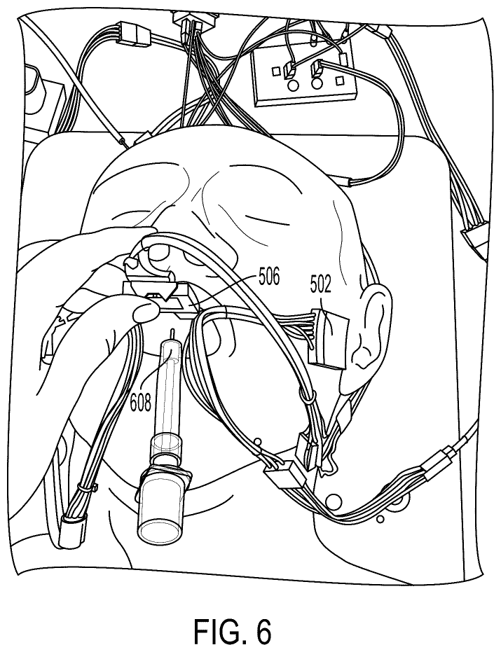

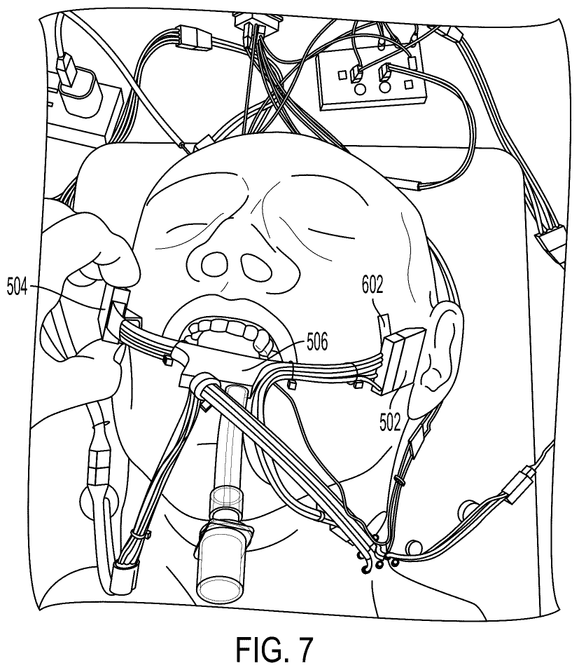

In describing an exemplary deployment of TIGER guard, one first considers placement of RF tags on a user/patient intubated with an endotracheal tube. Under an embodiment, an RF tag (FIG. 6, 608) is placed on the endotracheal tube itself (or integrated therein) and at one or more other positions on the skin surface of the corresponding user of the tube. As seen in FIG. 7 (602) an RFID tag is attached to a user's skin over a portion of check bone in proximity to the ear. Under this embodiment, another RF tag (not shown) is positioned on the patients skin in the same location opposite RF tag 602, FIG. 7. (Note that the central RFID tag can be attached to the endotracheal tube using various fastening mechanisms including tape or other adhesives. The central RF tag can be integrated into the body of the tube. In either case the central RF tag does not interfere with airflow through the tube. The RF tags may be attached to skin using any form of medical-grade, skin-friendly adhesive, often available as tapes, patches, or liquid adhesives).

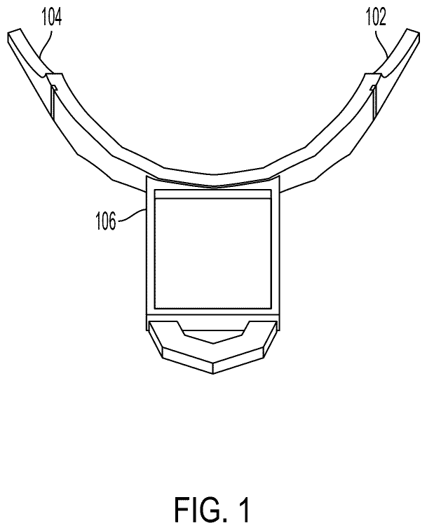

The TIGER guard structure is configured to place RFID probes in proximity to the RF tags described above. FIG. 1 shows a top view of the TIGER guard mechanical structure, under an embodiment. RFID probes are attached to the guard at opposing terminal ends (102, 104) of the guard arch and at the center point (106) of the guard arch. The probes may be attached to these locations using any form of fastening mechanism including adhesives, Velcro, etc. Under an alternative embodiment, the probes may be integrally formed as part of the guard. As yet another example, the guard may be configured to receive the probes into slots or recesses via press fit.

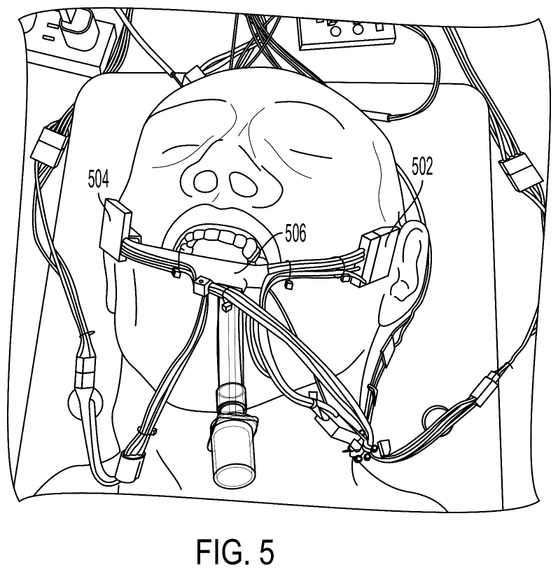

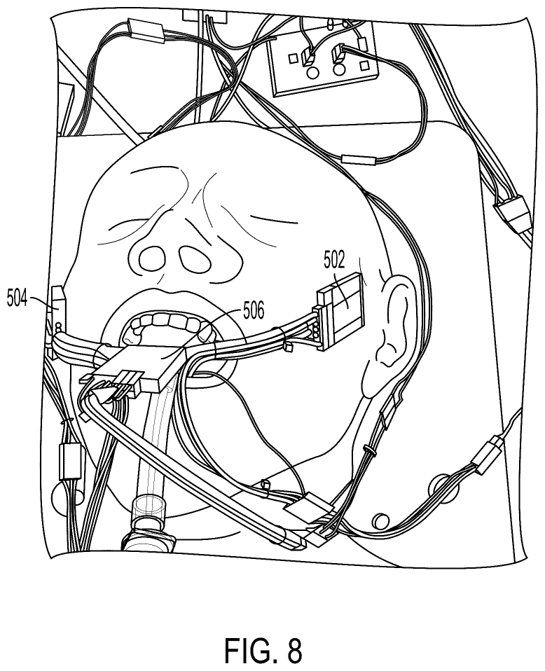

In operation, the guard locates and secures RFID probes at one or more locations in proximity to corresponding RF tags. FIGS. 5-8 illustrate a deployment of the TIGER guard.

Under this embodiment, probes (502, 504) are secured to the opposing terminal ends of the guard arch and probe 506 is secured to the guard arch at its center point. The guard is then placed on the patient. The center point of the arch and therefore the central probe 506 rest directly over an RF tag secured or affixed to the endotracheal tube. Alternatively, this endotracheal or “central” RF tag can be placed within the parioral region, e.g., adjacent the central upper lip or adjacent the central lower lip. The guard positions the terminal end probes (502, 504) over corresponding tags located on the face of the patient. Under one embodiment, terminal ends of the arch bias corresponding probes (502, 506) toward respective RF tags.

As already indicated above, FIGS. 5-8 illustrate a deployment of the TIGER guard. As seen in these figures, central probe 506 which is integrated into or otherwise secured to the guard at its central point is not itself directly affixed to the endotracheal tube but rather rests upon it.

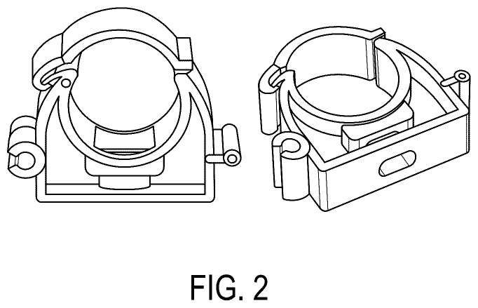

Alternatively, the central probe is directly attached to the tube. The probe can be secured using any fastening mechanism including adhesives, velcro attachments, clamps etc. As an example, FIG. 2 shows a mounting clip. The central probe can be attached to this clip which then clamps onto the tube. The clamp fixes the location of the central probe on the tube. Alternatively, the clamps may allow linear movement of the tube through the clamp itself.

As demonstrated in FIGS. 5-8, the deployed TIGER guard positions the RFID probes (502, 504, 506) over or in close proximity to corresponding RFID tags. Any movement of the patient or any other force or activity that initiates tube migration may cause reorientation of the probes relative to respective RF tags. Each probe is constantly monitoring the corresponding tag in real time. If the received RSSI strength of signal detected by a probe falls below a threshold value, the probe communicates the event to a microcontroller. The microcontroller may then visually display notice of the event or communicate it to applications running on one or more remote servers.

In a passive RFID system, RSSI (Received Signal Strength Indicator) is a measurement reported by the reader that indicates the power level of the backscattered signal it receives from a passive tag. The tag itself does not have a power source, so it relies on energy from the reader's signal to power its microchip and reflect a modulated signal back to the reader.

The RSSI value is typically measured in decibels per milliwatt (dBm). Since the tag uses backscattering, the RSSI is a fraction of the power the reader initially transmitted. The closer the tag is to the reader antenna, the stronger the return signal and the higher the RSSI value will be (less negative).

A high RSSI value generally correlates to a shorter distance between the reader and the tag.

To improve accuracy of RSSI readings, more complex algorithms combine RSSI with other data, such as:
Read count: The number of times a tag is successfully read per second.
Phase difference: A technique that uses the phase angle of the returned signal to determine a tag's location more reliably.
Filtering: Techniques like Kalman filters can help smooth out the noisy RSSI data to produce more stable readings
   Also note that the gain of the probe may be altered to adjust sensitivity of the probe at a given distance to a tag.

FIGS. 6 and 7 illustrate manual movement of the guard for purposes of demonstrating operation of the TIGER Guard monitoring system. FIG. 6 shows the potential effect of tube migration or any other movement which changes position of the RF probes relative to the RF tags. In particular with respect to FIG. 6, as RF probe 506 separates from the endotracheal RF tag 608, the probe detects a drop in RSSI signal strength below a threshold and sends information of the decoupling event to a microcontroller which then communicates with hardware to provide a visual alert. With respect to FIG. 7, as RF probe 502 separates from the corresponding RF tag 602, the probe detects a drop in RSSI signal strength below a threshold and sends information of the decoupling event to a microcontroller which then communicates with hardware to provide a visual alert.

Again note that any one or combination of tube migration, movement of the patient, an external force, and any detachment of central probe/face bar from the endotracheal tube (by passive or active decoupling or other failure of fastening mechanism) causes reorientation of the RFID probes relative to the respective RFID tags. This reorientation of RF probe relative to RF tag may trigger an alert given detected weakened RSSI signal strength as described above.

Also note that individual, all, or any combination of RFID probe/tag couplings can be can be required to trigger an alarm.

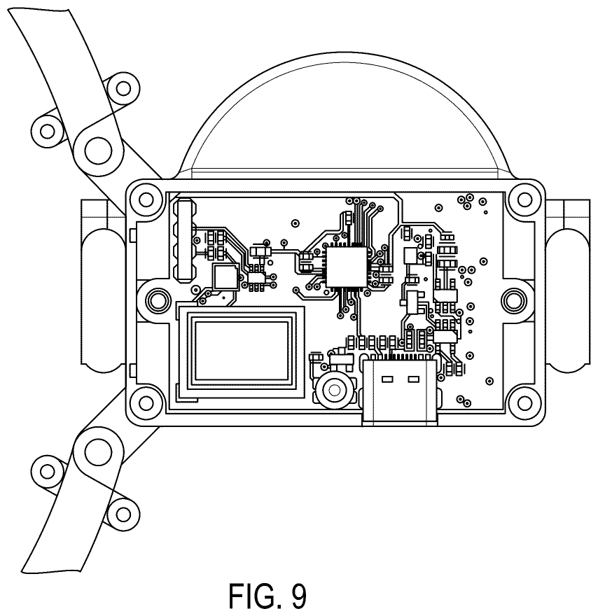

FIGS. 9-11 shows an integrated RFID microcontroller and guard structure, under an embodiment. The Tiger guard arch is analogous to the guard described above with respect to FIGS. 1-8. Two probes (912, 914) are located at opposing terminal ends of the guard arch while another central probe (916) is located at a central point of the arch. Note that the central probe is integrated into the microcontroller at a position analogous to the central probe described above. When deployed and attached to the endotracheal tube, the probes are located above or in proximity to corresponding RF tags in positions analogous to the embodiment described above.

The guard is integrally formed with a control box which itself incorporates a microcontroller, under an embodiment. The microcontroller is communicatively coupled (either wired or wirelessly) to the probes. A display is attached to the microcontroller. When any of the probe(s) detect a “detachment” event, the probe(s) provide this information to the microcontroller which displays a warning using right, middle, and left radio indicator buttons.

The microcontroller and probes may comprise arduino boards but embodiments are not so limited.

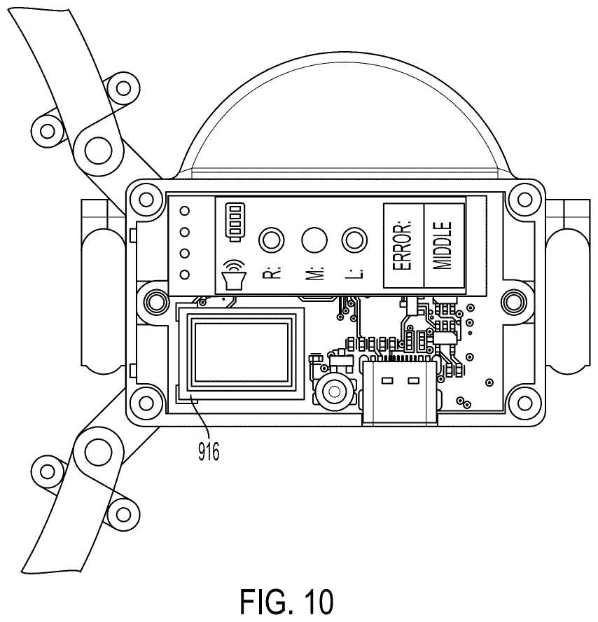

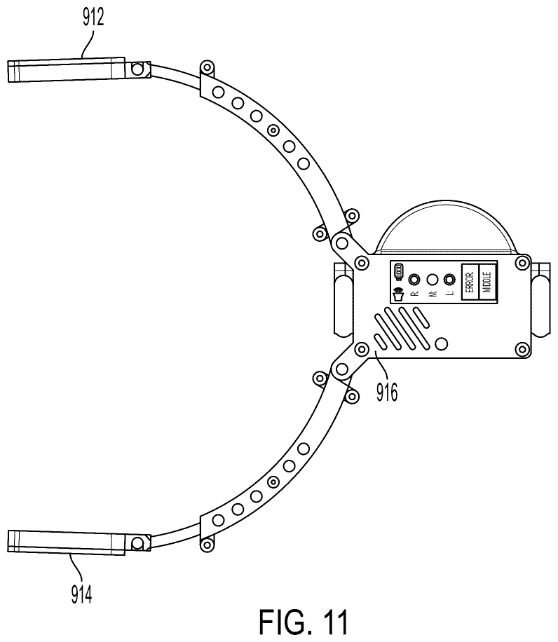

FIGS. 10 and 11 feature attachment clips at both ends of the control box. The attachment clips comprise c clamps or snap rings. When the TIGER guard is deployed, downwardly facing openings of the clamps may snap onto the endotracheal tube which then position the entire guard properly. These clamps or rings fix the location of the control box (and therefore central probe 916) on the tube. Alternatively, the clamps or snaps may allow linear movement of the tube through the clamps or rings themselves.

An important feature of TIGER's design is that the RFID tag is positioned externally to the inner space of the tube. This strategic placement ensures that the RFID technology does not compromise the airflow through the tube. By keeping the RFID components separate from the critical internal space of the tube, TIGER maintains the integrity of the ventilation process, eliminating any risk of interference or obstruction. Furthermore, the integration of the RFID detector into the face guard is designed with a keen consideration for practicality. This integration is carefully engineered to avoid interference with any medical procedures or devices in use during mechanical ventilation. The seamless coexistence of TIGER with existing medical equipment underscores its user-friendly nature, allowing healthcare providers to focus on patient care without concerns about technological hindrance. In summary, TIGER's RFID approach ensures both precision and non-intrusiveness. The external placement of the RFID tag and probe integration into the face guard exemplify the commitment to maintaining the functionality of the ventilation system while providing an effective solution to prevent potential complications associated with tube displacement.

This technology is a reliable defense mechanism against potential complications, such as oxygen desaturation, which can arise when the airway is mechanically ventilated. The RFID tags integrated into the tube communicate with the system in real-time, allowing for immediate detection of any movement outside the predetermined range. The value of TIGER lies in its simplicity and effectiveness. By providing a timely alert when tube displacement is detected, it empowers healthcare providers to take swift action, preventing respiratory complications and ensuring patient safety. Its user-friendly interface and seamless integration into existing equipment make it a practical tool for healthcare settings, enhancing the overall efficiency of medical professionals in monitoring and responding to critical situations. In essence, TIGER is a straightforward yet impactful technological advancement that prioritizes patient safety during mechanical ventilation by addressing a common and potentially serious issue.

This method uses a simple RFID approach to track the position of the tube after fixation using a single or multiple RFID detectors to localize the RFID tag(s) so that inappropriate translocation or migration of the tube is detected and triggers an alarm.

The RFID tag can be attached to the tube or it can be manufactured into the tube wall. The RFID tag can also be attached to any tube by a locking ring whose position can be adjusted before locking. This does not interfere with ventilation or restrict airflow in any way.

It should be noted that the RFID probe/tag locations are not limited to those detailed in the embodiments described herein. Further, additional or fewer probes may be used.

TIGER holds several competitive advantages that set it apart as a cutting-edge device for addressing the challenges associated with endotracheal tube displacement during mechanical ventilation:
1. Proactive Monitoring with Real-Time Alerts: TIGER'RFID-based system enables proactive monitoring of the tube's position in real-time. This immediate detection of any movement beyond the specified range allows for swift intervention, reducing the risk of oxygen desaturation and other complications associated with tube displacement.
2. Non-Intrusive RFID Technology: The external placement of the RFID tag on the tube ensures that the technology does not compromise the airflow within the tube's inner space. This design feature is a significant advantage as it allows for continuous monitoring without interfering with the core function of the ventilation system.
3. User-Friendly Integration: TIGER seamlessly integrates into existing medical equipment, ensuring compatibility with various ventilation systems. Its user-friendly design minimizes disruptions to medical procedures, allowing healthcare providers to focus on patient care without the burden of adapting to a complex or cumbersome technology.
4. Simple and Reliable Alarm System: The straightforward alarm circuit triggered by the RFID probe provides a reliable and immediate alert in the event of tube displacement. The simplicity of the alarm system enhances its effectiveness, ensuring that healthcare professionals can respond promptly to potential issues without the need for complex interpretations.
5. Enhanced Patient Safety: TIGER's ability to prevent oxygen desaturation by addressing tube displacement contributes significantly to patient safety during mechanical ventilation. Timely alerts empower healthcare providers to take corrective actions promptly, minimizing the risk of respiratory distress and related complications.
6. Versatility and Adaptability: TIGER's design allows for easy integration into various medical settings and ventilation setups. Its adaptability makes it a versatile solution applicable in diverse healthcare environments, ranging from intensive care units to emergency rooms.
7. Cost-Effective Prevention: By preventing complications associated with tube displacement, TIGER can contribute to cost savings in healthcare. Avoiding oxygen desaturation events and related complications may reduce the need for additional medical interventions, thereby potentially lowering overall healthcare costs.

Under an embodiment, the TIGER guard system is implemented using a greater number or fewer number of RF probe/tag couplings whereby the couplings may be placed at additional, fewer, and/or differing locations than those described above.

The Tiger guard face bar may comprise any material suitable for medical devices. Examples include polymeric or metallic materials but embodiments are not so limited.

The TIGER guard system described herein is implemented using passive RFID tags. Under an embodiment, the TIGER guard system uses powered RFID tags.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The Tube Interface Guard for Extubation and Ranging can be a component of a single system, multiple systems, and/or geographically separate systems. The Tube Interface Guard for Extubation and Ranging can also be a subcomponent or subsystem of a single system, multiple systems, and/or geographically separate systems. The components of Tube Interface Guard for Extubation and Ranging can be coupled to one or more other components (not shown) of a host system or a system coupled to the host system.

One or more components of the Tube Interface Guard for Extubation and Ranging and/or a corresponding interface, system or application to which the Tube Interface Guard for Extubation and Ranging is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the Tube Interface Guard for Extubation and Ranging can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the Tube Interface Guard for Extubation and Ranging and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the Tube Interface Guard for Extubation and Ranging and corresponding systems and methods include: microcontrollers with memory (such as electronically erasable programmable read only memory (EEPROM)), embedded microprocessors, firmware, software, etc. Furthermore, aspects of the Tube Interface Guard for Extubation and Ranging and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to. ” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the Tube Interface Guard for Extubation and Ranging is not intended to be exhaustive or to limit the systems and methods to the precise forms disclosed. While specific embodiments of, and examples for, the Tube Interface Guard for Extubation and Ranging and corresponding systems and methods are described herein for illustrative purposes, various equivalent modifications are possible within the scope of the systems and methods, as those skilled in the relevant art will recognize. The teachings of the Tube Interface Guard for Extubation and Ranging and corresponding systems and methods provided herein can be applied to other systems and methods, not only for the systems and methods described above.

The elements and acts of the various embodiments described above can be combined to provide further embodiments. These and other changes can be made to the Tube Interface Guard for Extubation and Ranging and corresponding systems and methods in light of the above detailed description.

1. A device comprising,
an arcuate face bar, wherein the arcuate face bar is configured for attachment to an endotracheal tube deployed upon a patient;
at least two radio frequency identification (RFID) probes secured to opposing terminal ends of the arcuate face bar, wherein the attachment of the arcuate face bar to the endotracheal tube positions the at least two RFID probes at locations in proximity to respective first and second RFID tags affixed to skin of the patient;
the RFID probes configured to communicate an occurrent of an event when one of the at least two RFID probes detects a received signal strength of a respective RFID tag below a threshold value.

2. The device of claim 1, wherein at least one of tube migration, movement of the patient, an external force, or detachment of the arcuate face bar from the endotracheal tube causes reorientation of the at least two RFID probes relative to the respective RFID tags.

3. The method of claim 1, wherein the at least two RFID probes comprise a first RFID probe.

4. The method of claim 3, wherein the attachment locates the first RFID probe in proximity to the first RFID tag affixed to cheek bone skin surface adjacent the patient's auris dexture ear.

5. The method of claim 1, wherein the at least two RFID probes comprises a second RFID probe.

6. The method of claim 5, wherein the attachment locates the second RFID probe in proximity to the second RFID tag affixed to cheek bone skin surface adjacent the patient's auris sinistra ear.

7. The method of claim 1, wherein the at least two RFID probes comprises a third RFID probe.

8. The method of claim 7, wherein the attachment locates the third RFID probe in proximity to a third RFID tag at least one of affixed to or integrated into the endotracheal tube without obstructing airflow therethrough.

9. The method of claim 7, wherein the attachment locates the third RFID probe in proximity to a third RFID tag affixed to a skin surface of the patient's parioral region.

10. The method of claim 9, wherein the location of the third RFID tag comprises skin surface adjacent to the patient's lower central lip.

11. The method of claim 9, wherein the location of the third RFID tag comprises skin surface adjacent to the patient's upper central lip.

12. The method of claim 1, wherein the at least two RFID probes emit a radio frequency signal into an environment of the respective RFID tag.

13. The method of claim 12, wherein the respective RFID tag's antenna captures this electromagnetic energy, which is used to power up a microchip inside the tag.

14. The method of claim 13, wherein the powered microchip modulates its stored identification data and sends it back to the respective at least two RFID probes as a return signal.

15. The method of claim 14, wherein the return signal comprises backscatter communication.

16. The method of claim 15, wherein the at least two respective RFID readers receive and decode the signal to identify the respective tag and process the information.

17. The method of claim 16, wherein the processing comprises detecting the received signal strength of the signal.

18. The method of claim 1, wherein the attachment comprises securing a central portion of the arcuate face bar to the endotracheal tube.

19. The method of claim 1, wherein the securing comprises use of an adhesive.

20. The method of claim 1, wherein the securing comprises use of a mounting clamp.
